(19) United States
(12) Patent Application Publication
(10) Pub. No.: US 2025/0277807 A1
(43) Pub. Date: Sep. 4, 2025

(54) SYSTEM AND METHOD MONITORING FLUID FLOW

(71) Applicant: DEKA Products Limited Partnership, Manchester, NH (US)

(72) Inventor: David Blumberg, JR., Epsom, NH (US)

(21) Appl. No.: 18/808,148

(22) Filed: Aug. 19, 2024

Related U.S. Application Data
(63) Continuation of application No. 63/533,773, filed on Aug. 21, 2023.

(51) Int. Cl. G01P 13/00

(52) U.S. Cl. CPC G01P 13/0006

(57) ABSTRACT

A system and method for monitoring fluid flow. The system of the present teachings includes a fluid flow monitor, where the fluid flow monitor is a Resonator Probe Antenna or a low-pass filter. The system includes placing the fluid flow monitor against a fluid compartment and measure the volume of fluid present in the fluid compartment to determine what stage of flow the fluid is in and monitor the flow. The system includes a controller involved in computing the volume of fluid based on a level of phase shift. The system includes disposable and durable parts.

### CROSS REFERENCE TO RELATED APPLICATIONS

This Application is related to and claims priority from U.S. Prov Application 63/533,773, filed Aug. 21, 2023 (AB077);

the contents of which are incorporated herein by reference.

### BACKGROUND

The present disclosure relates to monitoring the flow of a fluid to determine which stage of flow the fluid is in. In systems and methods involving the flow of fluid, it is often important that the flow be regulated, whether it be to maintain functionality, reduce waste, or to obtain precise flow control. A regulated fluid flow is a way of ensuring that the different aspects of the relevant system are functioning properly. Precise monitoring fluid flow is especially important when the fluid is flowing in a perfusion process into and through an organ. Human tissues are very sensitive to environment changes, so it is imperative that when fluids are passing through an organ, that fluid is being closely monitored to ensure protection of the tissue. The type of pump system likely determines the available methods for monitoring flow. This disclosure, for example, relates to pneumatic pumps. In a pneumatic pump system there is typically no direct way of monitoring the flow of a fluid to determine, for example, when an end of pump stroke has been reached. Presently, methods for monitoring fluid flow in a pneumatic pump system include the use of flow sensors in the fluid path and other similar methods.

In a system that employs disposable components along the entire fluid flow path, the use of in path flow sensors present a problem. Flow sensors are typically expensive, making it difficult for their inclusion in a system with disposable elements. In a system that involves a human tissue, the fluid being pumped through the system will contaminate elements of the system which, in turn, requires those elements to be replaced or thoroughly sterilized after each use. In such a system, it is preferable for the system to comprise disposable elements, adding a flow sensor to such a system would introduce a cost associated with the continual disposal and replacement of components with in path flow sensors.

What is needed is a system for monitoring fluid flow by externally detecting the amount of fluid present in a chamber, where the fluid path elements can be disposable and where the flow detection elements can accurately monitor fluid flow from an external position in a cost effective manner. What is needed is system for monitoring fluid flow where the fluid monitoring element is integrated into an external system and configured to monitor flow passing through a disposable pumping element. Being able to integrate such a detection scheme into an external system prevents the system for monitoring fluid flow from contacting potential contaminates contained within a system that involves some sort of fluid flow. What is needed is a method for monitoring fluid flow that remains cost effective while also remaining accurate.

### SUMMARY

In accordance with some configurations, the present teachings include a system and method for monitoring fluid flow. Among other features, the system of the present teachings can include a pump body which may be fluidly connected with a pump subsystem. In an aspect, the fluid being pumped is a dielectric fluid. In an aspect the dielectric fluid is, for example but not limited to, water, blood, and preservation solution. In an aspect, the pump subsystem is from an external apparatus. In an aspect the pump subsystem can pump the dielectric fluid through a tissue, the tissue can be, for example but not limited to, a kidney.

The system of the present disclosure includes a pump body. The pump body being fluidly connected with a circulation system and a pump subsystem which cooperate with one another to pump the dielectric fluid through the circulation system. The pump subsystem drives elements of the pump body to create fluid flow. The pump body further includes at least one fluid compartment which is located along the flow path of the fluid within the circulation system. The system further includes a flow monitor that is operatively coupled with the fluid compartment for the purpose of detecting the amount of fluid in the fluid compartment. By detecting the amount of fluid in the fluid compartment the flow monitor can properly measure the flow of the fluid and detect when a stroke has been completed.

In an aspect of the system of the present disclosure the flow monitor is a Resonator Probe Antenna configuration for monitoring the flow of the dielectric fluid. In one arrangement the Resonator Probe Antenna configuration has a transmit antenna and a receive antenna. The Resonator Probe Antenna configuration also has a receive antenna. In some embodiments, the transmit antenna and receive antenna is set to a frequency of 0.895 GHz. The receive antenna and transmit antenna are located within close proximity of one another, forming a transmit/receive sensor. The Resonator Probe Antenna configuration further includes connecting the sensor to a phase/gain measurement module. In an aspect the sensor is placed against an external surface of the fluid compartment. This positions the sensor close to an element of the flow path, allowing for an ideal location to operationally detect the amount of fluid within the fluid compartment. When fluid is being pumped through the system and subsequently through the fluid compartment, the transmit antenna can propagate a wave which passes through the fluid and returns to the receive antenna. The fluid, being a dielectric fluid, will affect the velocity at which a propagation is able to travel from the transmit antenna to the receive antenna. The dielectric constant (εr) of the fluid in combination with the fluid volume will determine the impact on the velocity of the propagation. In a system such as this, where the volume of fluid within the fluid compartment is fluctuating, the εr functions as a relative dielectric constant (εre). The εre of the fluid will change depending on the identity of the fluid and the volume of the fluid within the fluid compartment at any moment in time. The phase velocity of a propagation from said transmit antenna in the absence of an interference is a known variable. When the dielectric fluid is introduced, and subsequently slows down the propagation, the velocity is changed, resulting in a phase shift of the propagation. In the phase/gain measurement module analyzes the phase shift, and calculates a volume of fluid in the fluid compartment based on the phase shift, allowing a determination of the change in volume and ultimately the flow. The larger the phase shift, the larger the volume of fluid in the fluid compartment, and the lack of a phase shift indicates a lack of fluid in the fluid compartment. By determining the volume of fluid in the fluid compartment, the system monitors the flow of fluid and determines what phase a stroke is in.

In an aspect the pump body is a disposable piece of equipment and the sensors of the Resonator Probe Antenna configuration are integrated with the pump body. The sensors are connected to the exterior surface of the pump body and have connected coaxial cable attached to allow the sensors to be connected to the phase/gain measurement module. The intention would be to utilize the pump body in an external apparatus so that the system may be completed. Then when a pump body has been used, it may be discarded and replaced with another pump body for subsequent completions of the system of the present disclosure.

In an aspect the pump body is a disposable piece of equipment and the Resonator Probe Antenna configuration is located externally from the pump body. In this embodiment the sensors of the Resonator Probe Antenna configuration are integrated into latching arms. A pump body will be placed into a terminal adapter and the clamps will hold the pump body in place while by clamping over the fluid compartments, while simultaneously pressing the sensors against the fluid compartments so that the flow of the fluid may be monitored and the sensors can properly send propagations through the fluid compartment. In this embodiment, after each use the pump body will be discarded and replaced, but the Resonator Probe Antenna configuration will remain. This eliminates the need to have disposable elements that include the sensor configuration that result in disposing of the sensor along with the disposable elements. This system separates the Resonator Probe Antenna configuration from the disposable element, allowing for reuse while also preventing user interaction by proximity with the fluid effective dielectric.

The system of the present disclosure provides an embodiment where the flow monitor is a low-pass filter phase shift network configuration for monitoring the flow of the dielectric fluid. In an aspect, the low-pass filter phase shift network configuration has two capacitor plates. In as aspect the low-pass filter may be for example, but not limited to, a third order filter. In an aspect, the two capacitor plates are connected by an inductor. In an aspect, the two capacitors have a reciprocal capacitor ground plate. The capacitor ground plate has a reference voltage. The low-pass filter phase shift network configuration further includes connecting the capacitor plates to a phase/gain measurement module. In an aspect the dielectric fluid completes the low-pass filter by serving as the dielectric component of the capacitor plates. In an aspect the capacitor plates and capacitor ground plates are placed against the external surface of the fluid compartment. This keeps the plates in close proximity to the flow of the fluid, allowing the low-pass filter to be completed with the dielectric fluid. When fluid is being pumped through the system and subsequently, the fluid compartment, the dielectric fluid, along with the ground plate and capacitor plates, forms two capacitors. The changing flow of fluid will result in a change in the volume of fluid in the fluid compartment. As a result, the capacitance of the capacitors will change as the dielectric component (dielectric fluid) of the capacitors changes. Along with a change in capacitance comes a band width cut-off change. The change in band width cut-off is indicative of the level of fluid in the fluid compartment. When the dielectric fluid is introduced, and subsequently increases the capacitance, the low-pass filter cut-off frequency is changed, resulting in a transmission phase shift. In the phase/gain measurement module is a controller, which analyzes the phase shift, indicating the volume of fluid in the fluid compartment, allowing the user to monitor the flow. The larger the phase shift, the larger the volume of fluid in the fluid compartment. By determining the volume of fluid in the fluid compartment the user is able to monitor the flow of fluid and determine what phase a stroke is in.

In an aspect the pump body is a disposable piece of equipment and the capacitor plates and capacitor ground plates of the low-pass filter phase shift network configuration are integrated with the pump body. The capacitor plates and capacitor ground plates are connected to the external surface of the pump body and have connected coaxial cables attached to allow the plates to be connected to the phase/gain measurement module. The intention would be to utilize the pump body in an external apparatus so that the system may be completed. Then when a pump body has been used, it may be discarded and replaced with another pump body for subsequent completions of the system of the present disclosure.

In an aspect, the pump body is a disposable piece of equipment and the low-pass filter phase shift network configuration is located externally from the pump body. In this embodiment the capacitor plates and capacitor ground plates of the low-pass filter phase shift network configuration are integrated into latching arms. A pump body will be placed into a terminal adapter and the latching arms will hold the pump body in place while by clamping over the fluid compartments, while simultaneously pressing the low-pass filters against the fluid compartments so that the flow of the fluid may be monitored and the capacitor plates can properly interact with the fluid inside of the fluid compartment. In this embodiment, after each use the pump body will be discarded and replaced, but the low-pass filter phase shift network configuration will remain. This eliminates the need to have disposable elements that include the low-pass configuration which would result in disposing of the low-pass filter along with the disposable elements. This system separates the low-pass filter from the disposable element, allowing for extensive reuse while also decreasing the likelihood of data interference from contact with the low-pass filter.

In an aspect, when the pump is being used to pump fluid through a tissue, the role of the perfusion loop is to replicate basic biological functions that would otherwise take place in the body. These include oxygenation, control of carbon dioxide, thermal control, and nutrient supply. Oxygenation and carbon dioxide control are conducted through the use of a membrane oxygenator. A heat exchanger is used to maintain desired perfusate temperature. The perfusion fluid leaves the tissue, is passed through an oxygenator, is passed through a heat exchanger, and is then pumped back into the tissue. Nutrients are suppled in the perfusion solution and can be added manually or through the use of automated infusion pumps. Output generated by the tissue flows out of the tissue and is available for sampling through sterile sample ports. Output can be directed back into the perfusion loop or discarded. Output flow rates and volumes are measured and stored by the system. In the event that recirculating output proves to be a challenge, the system can be modified such that the output is collected or potentially passed through a dialysis loop. In an aspect, when the dielectric fluid is blood, the blood can include whole blood or a diluted/modified/altered blood composition, for example. In some configurations, direct acting pneumatic pumps can be used in tandem to supply a continuous flow of perfusate. Direct acting pneumatic pumps can include active inlet and outlet valves so that flow in a perfusate flow circuit can be highly controlled.

### BRIEF DESCRIPTION OF THE DRAWINGS

The foregoing features of the disclosure will be more readily understood by reference to the following description, taken with reference to the accompanying drawings, in which:

FIG. 1 is a schematic perspective of a Resonator Probe Antenna;

FIGS. 2A-B are schematic perspectives of different embodiments of Resonator Probe Antennas on an external surface of a fluid compartment; FIGS. 2C-E are schematic cross-sections of a fluid compartment showing the various pump stroke phases;

FIGS. 3A-B are schematic perspectives of different embodiments of low-pass filters;

FIGS. 4A-B are schematic perspectives of different embodiments of low-pass filters on an external surface of a fluid compartment; FIG. 4C is a graph depicting phase shift in a low-pass filter;

FIGS. 5A-C are schematic perspectives of a latching arm;

FIGS. 6A-D are schematic perspectives of a terminal adapter configured to receive a pump body and latching arms;

FIG. 7 is a schematic perspective of a latching arm configured with an embodiment of a Resonator Probe Antenna;

FIG. 8 is a method of monitoring fluid flow;

FIG. 9 is a method of monitoring fluid flow; and

FIG. 10 is a graph depicting the use of resonator probe antennas against a commercial in-line flow sensor.

### DETAILED DESCRIPTION OF THE DRAWINGS

The system for monitoring fluid flow in accordance with the present disclosure is described in detail herein. Specifically, the system and method of the present teachings is configured to allow for the monitoring of fluid flow by determining the amount of fluid in a chamber and using that to detect a stage of the pumping stroke. The system of the present teachings includes, but is not limited to including, a set of disposable components and a set of durable components. The disposable components include, but are not limited to, a pump body, a fluid compartment and a sensor array. The system of the present teachings also includes flow monitors; the flow monitors may be a disposable or a durable component. The durable components include, but are not limited to including, a terminal adapter, latching arms. The system of the present teachings may involve connection with a pump assembly.

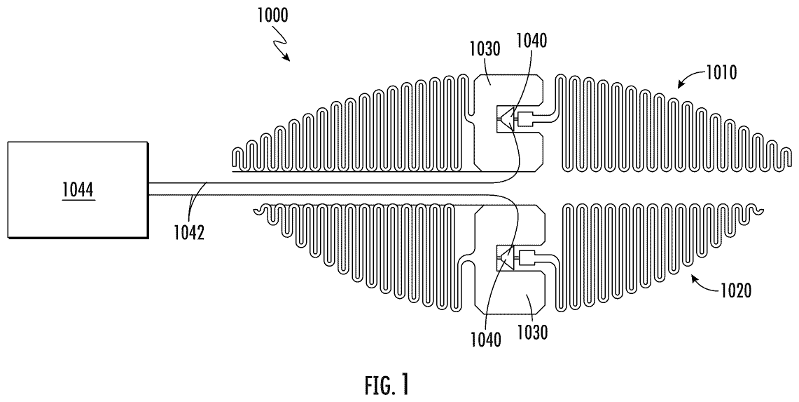

Referring now to FIG. 1, various embodiments of Resonator Probe Antennas that operate to detect fluid in a fluid chamber are illustrated. In an aspect, the system of the present teachings comprises a Resonator Probe Antenna 1000. In an aspect, the Resonator Probe Antenna 1000 has a transmit antenna 1010 and a receive antenna 1020. In an aspect, the Resonator Probe Antenna has a ground plate 1030 and electrical connectors 1040. In an aspect, the transmit antenna 1010 can be configured to resonate signals at a frequency of 895 MHz. In an aspect, the receive antenna 1020 can be configured to resonate at 895 MHz. In an aspect, the transmit antenna 1010 and receive antenna 1020 are connected to the ground plate. In an aspect, the electrical connectors 1040 are W.FL or X.FL connectors and are situated upon the ground plate 1030. In an aspect, the electrical connectors 1040 are further configured to interact with cables 1042, cables may be, for example but not limited to, 0.81 mm coaxial cables. In an aspect, through said cables the Resonator Probe Antenna 1000 is communicatively coupled with a phase/gain measurement module 1044. Said phase/gain measurement module is configured to receive and analyze data from the Resonator Probe Antenna 1000. Said phase/gain measurement module may further include a controller configured to compute the amount of fluid present in the fluid compartment based on the extent of phase shift or lack thereof.

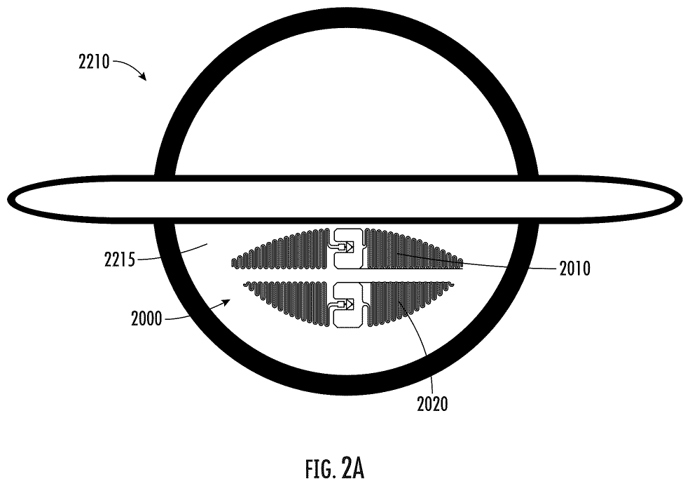

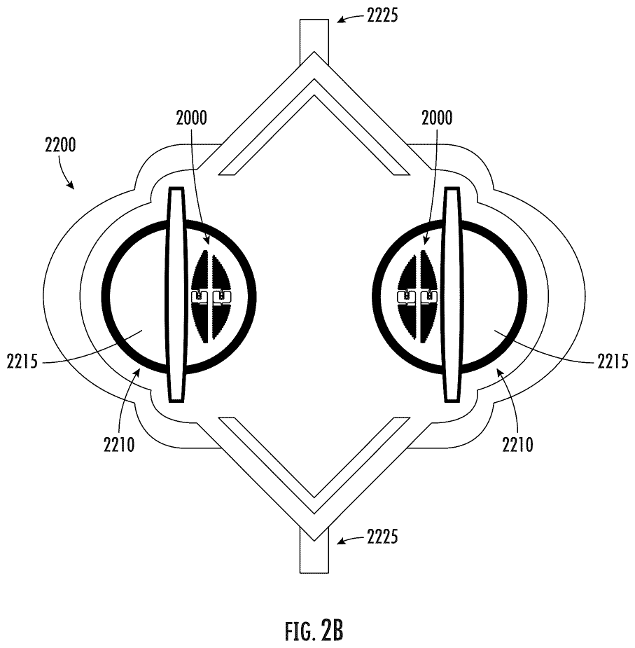

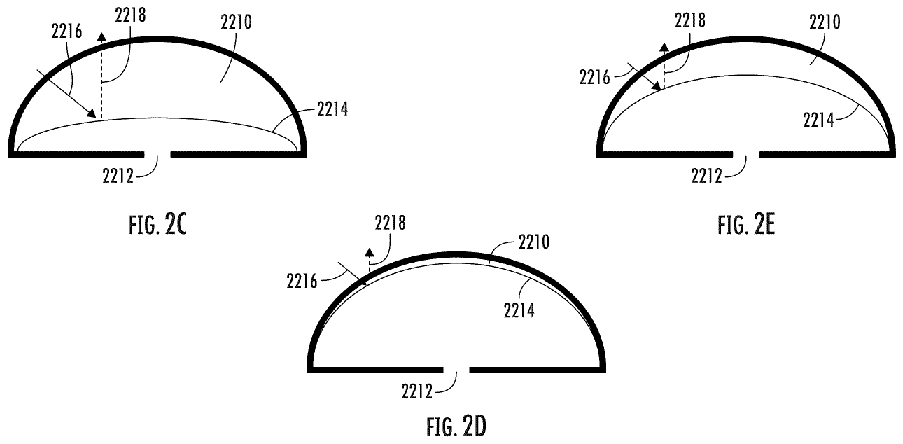

Referring now to FIGS. 2A-2E, various embodiments of Resonator Probe Antennas 2000 connected to the external surfaces 2215 of a pump body 2200 are illustrated. In an aspect, the Resonator Probe Antennas 2000 can be placed on an external surface 2215 of a fluid compartment 2210. In an aspect the fluid compartment 2210 is an element of a pump body 2200. In an aspect, the pump body 2200 has flow line connections 2225. In an aspect, the pump body may be connected to an external pump subsystem. For example, but not limited to the example, the pump body 2200 may be fluidly connected to a pump subsystem by the flow line connections. Further, the fluid compartments 2210 may be connected to the pump subsystem and may be connected to pneumatic pumps. To further illustrate the example, the pump subsystem may pneumatically pump fluid through the pump body where fluid will be pumped through the fluid compartments 2210. In an aspect, the fluid compartments 2210 are an element of the pump body 2200 where fluids pass through. In an aspect, the external surface 2215 of the fluid compartments 2210 is an area that can fit a flow monitor, so that that flow can be monitored on the actual path of the flow.

Still referring to FIGS. 2A-2E, in an aspect, Resonator Probe Antennas 2000 are of the exemplary embodiments described in FIG. 1. In an aspect, the Resonator Probe Antennas 2000 are configured to measure the amount of fluid in the fluid compartments 2210. This may be achieved, for example, but not limited to this example, by placing the Resonator Probe Antennas 2000 on the external surface 2215 of the fluid compartments 2210. To further illustrate the example, when fluids are pumped through the pump body 2200 and subsequently through the fluid compartments 2210, the fluid compartments 2210 will have varying amounts of fluid inside during the different stages of the fluid being pumped. As the amount of fluid in the fluid compartments varies, a transmit signal radiating from the transmit antenna 2010 will propagate through the dielectric fluid being passed through the fluid compartments 2210. The signal will then be passed by through the dielectric fluid and received by the receive antenna 2020. Because the fluid being passed through the fluid compartments 2210 is a dielectric fluid, the time it takes for a signal to pass from the transmit antenna 2010 through the fluid and back to the receive antenna 2020 will change based on the amount of fluid present in the fluid compartment 2210. Specifically, the more fluid present in the fluid compartment 2210, the longer it will take the signal to reach the receive antenna 2020. The resulting effect of this time change represents a shift in the insertion phase of the signal. The phase shift is measured and analyzed by the phase/gain measurement module. A controller in the phase/gain measurement module is configured to compute the amount of fluid present in the fluid compartment 2210 based on the extent of phase shift or lack thereof. This analysis illustrated in FIGS. 2C-2E provides information on the amount of fluid in the fluid compartment 2210. As the pump is driven pneumatically the controller directs air pressure into and out of the pump body via the pneumatic orifice 2212 to cause flexible membrane 2214 to expand and contract thereby changing the quantity of fluid within the fluid compartment. As seen at FIG. 2C, the stage of the pump is early in the stroke and there is a relatively large quantity of fluid within the fluid compartment 2210 and as the transmit signal 2216 propagates through the dielectric fluid it undergoes a large phase shift. In contrast, at FIG. 2E, the pump is mid-stroke and the fluid compartment 2210 contains less fluid such that the transmit signal 2216 undergoes less of a phase shift in the return signal 2218 due to the effective dielectric constant, thereby indicating less fluid in the fluid compartment 2210. Finally, as the pump reaches end of stroke as shown at FIG. 2D, there is little fluid in the fluid compartment causing a negligible amount of fluid within the fluid compartment 2210 causing a small insertion phase shift between the transmit signal 2216 and the return signal 2218 providing an indication of end of stroke. Determining the amount of fluid present in the fluid compartments 2210 indicates what pumping stage the fluid is flowing in and it will allow the user to ensure that the fluid is flowing properly. When pumping fluid through tissue, such as a kidney, it is very important that the flow is properly monitored because too much or too little flow could damage the tissue. To address this importance, the Resonator Probe Antenna 2000 detect the fluid present so that the flow can be monitored, and whatever the fluid is being passed through is kept maintained as well.

Referring specifically to FIG. 2B, which illustrates the pump bodies 2200 with a Resonator Probe Antenna 2000. In an aspect, the pump bodies 2200 are disposable. For example, but not limited to the example, having a disposable pump body 2200 allows a user to discard it after each use or after a certain number of uses. To further illustrate the example, if the pump body 2200 is being used in conjunction with a system involving bodily fluids, then it is convenient to discard the pump body 2200 after each use. In an aspect, the Resonator Probe Antennas 2000 are integrated into the disposable pump bodies 2200, and each subsequent disposable pump body 2200 will also have an integrated Resonator Probe Antenna 2000.

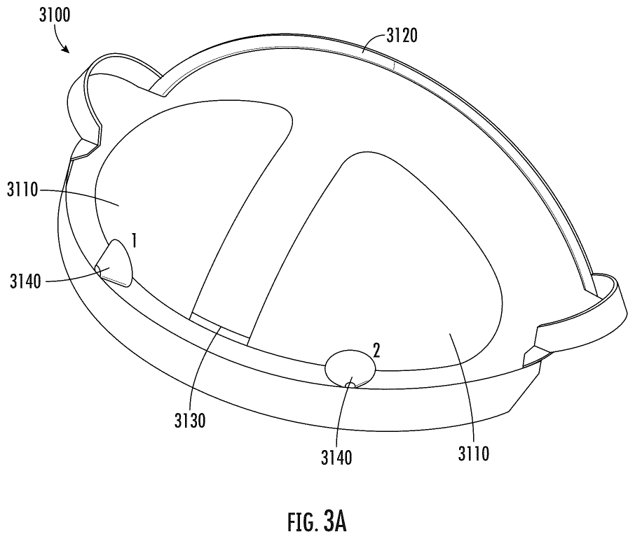

Referring now to FIGS. 3A-3B, various embodiments of low-pass filters 3100, 3101 are illustrated. In an aspect, the system of the present teachings comprises a third-order low-pass filter 3100, 3101. In an aspect, the low-pass filter has capacitor plates 3110. In an aspect, the low-pass filter 3100, 3101 has capacitor ground plates 3120. In an aspect, the low-pass filter has an inductor 3130. In an aspect, one capacitor plate 3110 is connected to another capacitor plate 3110 by an inductor 3130. In an aspect, a capacitor system is formed between the capacitor plates 3110 and the capacitor ground plate. In an aspect, the dielectric needed for a capacitor is the dielectric fluid that can flow through the pump body and into the fluid compartments. In an aspect, the capacitor plates 3110 are coupled with electrical connectors 3140. In an aspect, the electrical connectors 3140 are W.FL or X.FL connectors and they are situated upon the capacitor plates 3110. In an aspect, the electrical connectors 3140 are further configured to interact with cables, cables may be, for example but not limited to, 0.81 mm coaxial cables. In an aspect, through said cables the low-pass filter 3100, 3101 is communicatively coupled with a phase/gain measurement module. Said phase/gain measurement module is configured to receive and analyze data from the low-pass filter 3100, 3101. Said phase/gain measurement module is further coupled with a controller configured to compute the amount of fluid present in the fluid compartment based on the shift of the low-pass filter cut-off frequency, or lack thereof. FIG. 3A represents a first embodiment of the low-pass filter, FIG. 3B represents an alternative embodiment of the low-pass filter.

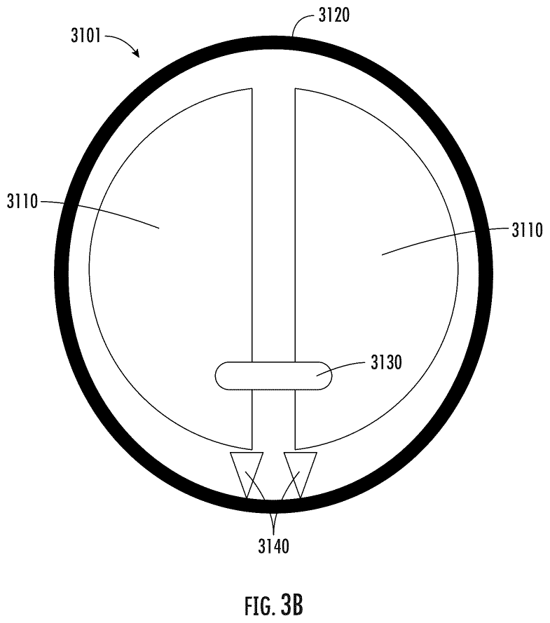

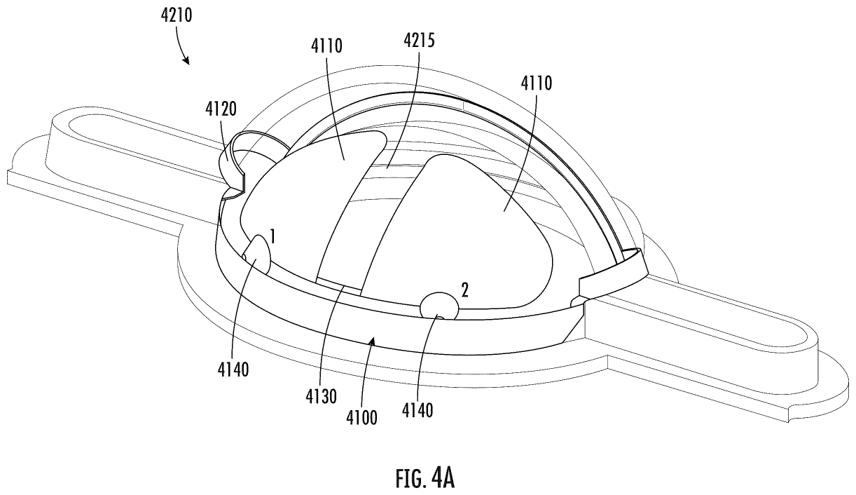

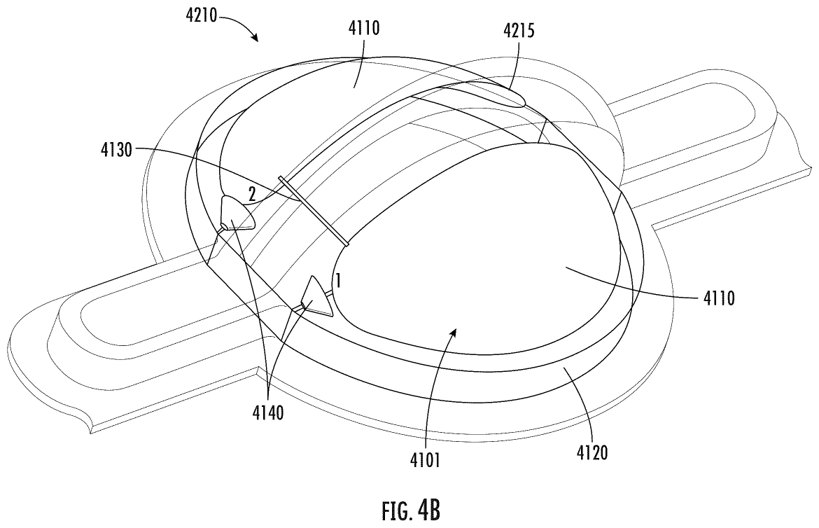

Referring now to FIGS. 4A-4C, various embodiment of low-pass filters 4100, 4101 connected to the external surface 4215 of fluid compartments 4210 are illustrated. In an aspect, the low-pass filters 4100, 4101 can be placed on an external surface 4215 of a fluid compartment 4210. In an aspect the fluid compartment 4210 is an element of a pump body. In an aspect, the pump body has flow line connections. In an aspect, the pump body may be connected to an external pump subsystem. For example, but not limited to example, the pump body may be fluidly connected to a pump subsystem by the flow line connections. Further, the fluid compartments 4210 may be connected to the pump subsystem and may be connected to pneumatic pumps. To further illustrate the example, the pump subsystem may pneumatically pump fluid through the pump body where fluid will have pumped through the fluid compartments 4210. In an aspect, the fluid compartments 4210 are an element of the pump body where fluids pass through. In an aspect, the external surface 4215 of the fluid compartments 4210 is an area that can fit a flow monitor, so that that flow can be monitored on the actual path of the flow.

Still referring to FIGS. 4A-4C, in an aspect, low-pass filters 4100, 4101 are of the exemplary embodiments described in FIGS. 3A-3B. In an aspect, the low-pass filters 4100, 4101 are configured to measure the amount of fluid in the fluid compartments 4210. This may be achieved, for example, but not limited to this example, by placing capacitor plates 4110 on the external surface 4215 of the fluid compartment 4210. The capacitor plates 4110 can then be connected to each other by an inductor 4130, to form the third order low-pass filter, and also be connected to a capacitor ground plate 4120. This would create a capacitor, with the dielectric fluid of the pump subsystem being the dielectric element of the capacitor. To further illustrate the example, when fluids are pumped through the pump body and subsequently through the fluid compartments 4210 will have varying amount of fluid inside during the different stages of the fluid being pumped, imparting a varying propagation reduction by effective dielectric constant variation. As the amount of fluid in the fluid compartment varies, the amount of the dielectric (dielectric fluid) in the low-pass filters 4100, 4101 will change. This change in the amount of the dielectric results in a low-pass cut-off frequency shift. This low-pass cut-off frequency shift is indicative of the amount of fluid in the fluid compartment 4210. Because band-cut off shift is caused by affecting the dielectric of a capacitor, when the fluid flowing through the fluid compartments 4210 changes, band width cut-off shift occurs because the fluid is the dielectric of the capacitors. A controller in the phase/gain measurement module is configured to compute the amount of fluid present in the fluid compartment 4210 based on the extent of band width cut-off shift or lack thereof. This analysis will present a user with information on the amount of fluid in the fluid compartment 4210. Determining the amount of fluid present in the fluid compartments 4210 will indicate to a user what stage the fluid is flowing in and it will allow the user to ensure that the fluid is flowing properly. When pumping fluid through tissue, such as kidneys, it is very important that the flow is properly monitored because too much or too little flow could damage the tissue. To address this importance, the low-pass filters 4100, 4101 detect the fluid present so that the flow can be monitored, and whatever the fluid is being passed through is kept maintained as well. As seen in FIG. 4C, which depicts a simulation of phase shift, there is a notable shift in a full fluid compartment 4210 compared to that of an empty fluid compartment 4210. In an aspect, in its application the low-pass filter will experience phase shift similar to that of the graph in FIG. 4C, and those values will be used to determine the amount of fluid present. In an aspect, cut-off frequency is determined by the function depicted in FIG. 4C.

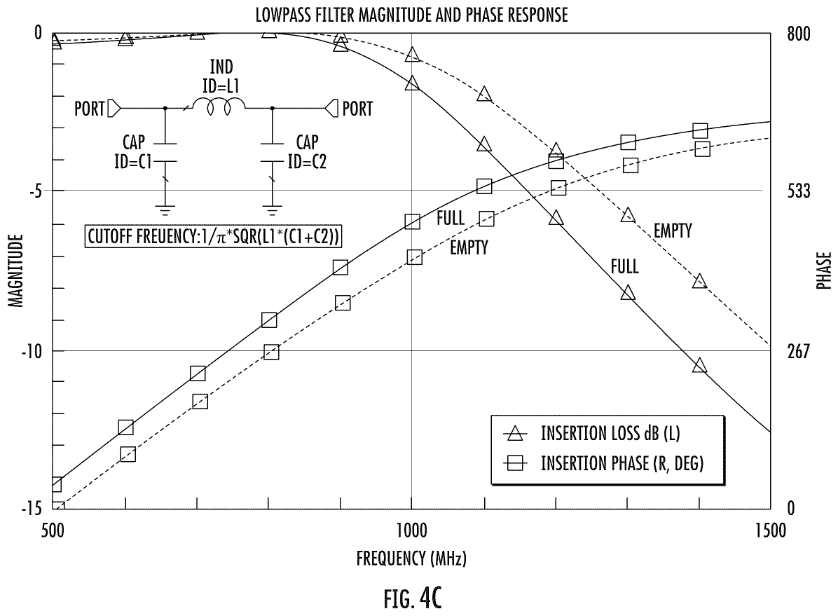

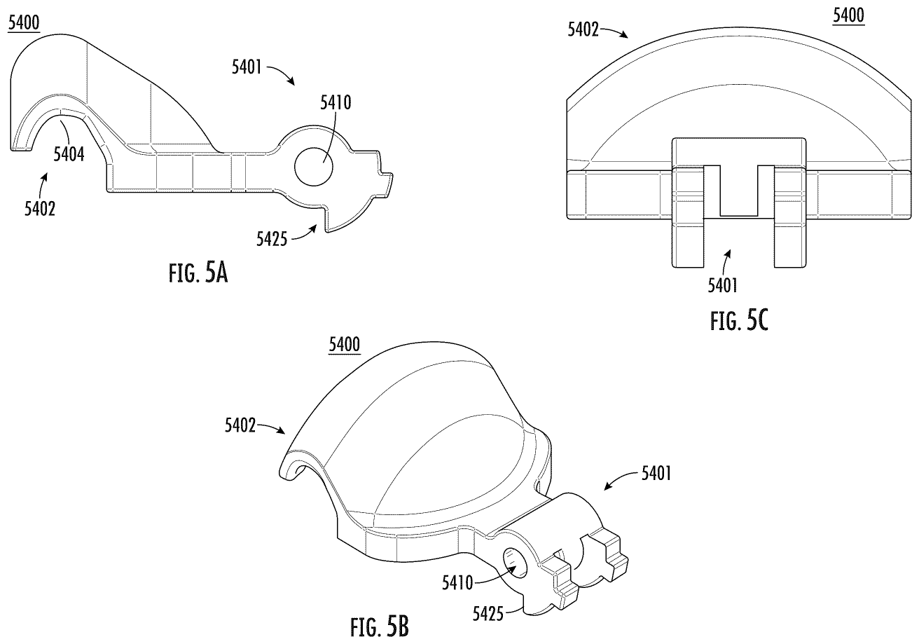

Referring now to FIGS. 5A-5C, which illustrate a latching arm 5400. In an aspect, the latching arms 5400 have a swivel portion 5401 and a clamping portion 5402. In an aspect, the swivel portion 5401 has a screw slot 5410. In an aspect, the clamping portion 5402 has a compartment press 5404. In an aspect, the latching arm 5400 has a retention detent 5425. In an aspect, the screw slot 5410 is configured to interact with a screw. In an aspect, the latching arm 5400 can swivel back and forth when a screw is engaged with the latching arm 5400. In an aspect, the compartment press 5404 is configured to engage with the external surface of the fluid compartments described in FIGS. 2A-2D an 4A-4D. In an aspect, the compartment press 5404 is configured exactly to match the external surface of the fluid compartment. In an aspect, a spring plunger will catch in the retention detent 5425 to hold the latching arm 5400 in place against the fluid compartment. This would allow for the latching arm 5400 to be used to lock the pump bodies in place by ensuring a tight fit between the compartment press 5404 and the fluid compartment. In an aspect, the latching arm 5400 may be configured to have an integrated flow monitor. In an aspect, the flow monitor may be a Resonator Probe Antenna or a low-pass filter. In an aspect, the flow monitor may be integrated into the compartment press 5404 of the latching arm 5400, so that it may be closed against the fluid compartment for purposes of monitoring fluid flow. Having the flow monitor integrated into the latching arm 5400 would allow for the flow monitors to be saved and used when the pump bodies are disposable. In an aspect, having the flow monitor integrated into the latching arm 5400 works to prevent user interaction by proximity with the fluid-effective dielectric constant.

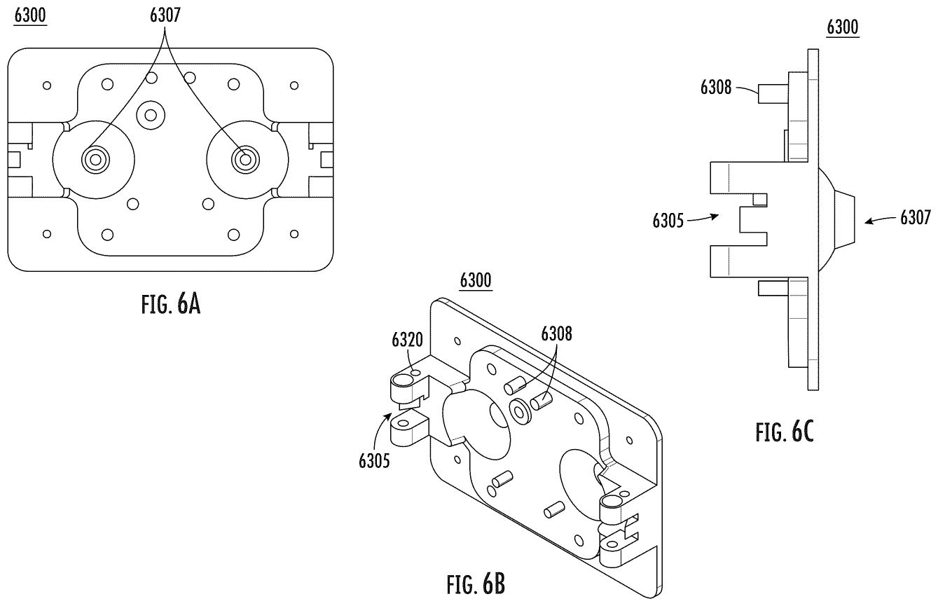

Referring now to FIGS. 6A-6D, which illustrate a terminal adapter 6300. In an aspect, the terminal adapter has compartment slots 6307. In an aspect, the terminal adapter 6300 has a latching arm slot 6305. In an aspect, the terminal adapter has pins 6308. In an aspect, the terminal adapter 6300 has a latching arm slot 6305. In an aspect, the terminal adapter 6300 has a plunger slot 6320. In an aspect, the terminal adapter is configured to receive a pump body, a pump body may be as described in 2C-2D. In an aspect, a pump body may be placed into the terminal adapter 6300. The pins 6308 from the terminal adapter 6300 fit into receptacles on the pump body, further, the fluid compartments from the pump body fit into the compartment slots 6307 of the terminal adapter 6300. These elements allow a pump body to be placed into a terminal adapter. This is especially helpful when the pump body is a disposable element, meant to be discarded and replaced after use. The terminal adapter 6300 can be fixed onto an external system with a pump system, and pump bodies can be placed into the terminal adapter 6300 and discarded and replaced while the terminal adapter 6300 remains in place in the external system.

Referring now specifically to FIG. 6D, which illustrates a terminal adapter 6300 with latching arms 6400. In an aspect, the latching arms 6400 are connected to the terminal adapter 6300. In an aspect the latching arms 6400 may be latching arms 6400 as described in FIGS. 5A-5C. This may be achieved, for example, but not limited to, lining up the screw slot 6410 of the latching arm 6400 with the latching arm slot 6305 of the terminal adapter 6300 and placing a shoulder screw 6310 through the matched up slots. A spring plunger 6325 placed through the plunger slot and a torsion spring 6315 placed on the latching arm 6400 creates a tight seal against the fluid compartment. When the latching arm 6400 is rotated and pressed against the fluid compartment, the spring plunger 6325 engages with the retention detent of the latching arm 6400 and locks it into place. In an aspect, a flow monitor may be integrated into the compartment press 6404 of the latching arm. In an aspect, the flow monitor may be a Resonator Probe Antenna or a low-pass filter. When the pump body is a disposable element, having the flow monitor integrated into the latching arm 6400 would eliminate the need of having the flow monitoring device on each pump body and having those be discarded. Having the flow monitor integrated into the latching arm 6400 may also prevent user interaction by proximity with the fluid effective dielectric constant.

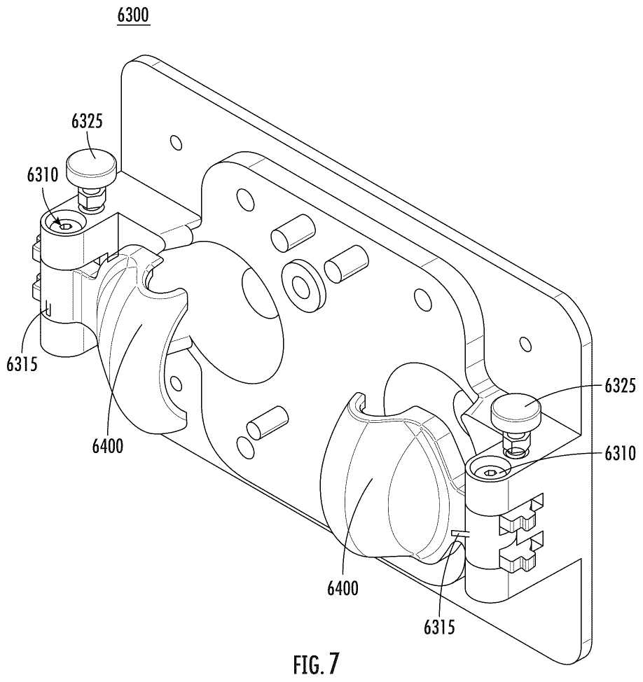

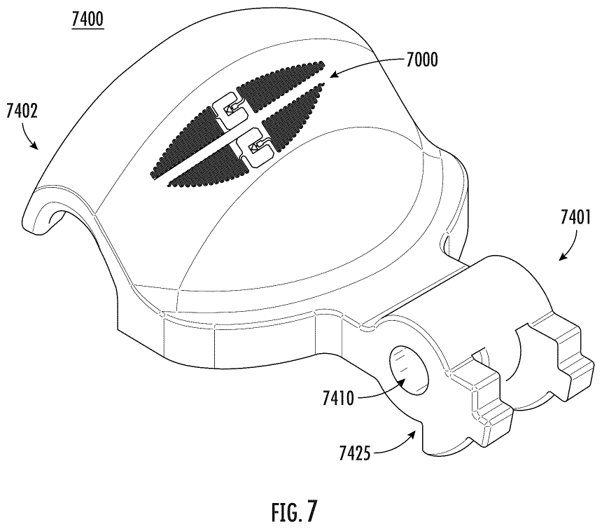

Referring now to FIG. 7, which illustrate a latching arm 7400 with an integrated flow monitor. In an aspect, the latching arms 7400 have a flow monitor integrated into the compartment press 7404 of the latching arm 7400. In an aspect, the flow monitor is a Resonator Probe Antenna 7000. In an aspect, the Resonator Probe Antenna 7000 is a Resonator Probe Antenna 7000 as described in FIG. 1. In an aspect, when a pump body is placed in the terminal adapter, the latching arms 7400 having a Resonator Probe Antenna are clamped down onto the pump body. In an aspect, the compartment press 7404 is configured to fit the fluid compartment and closes on top of it, being locked in placed by the spring plunger. The Resonator Probe Antennas 7000 are positioned in the compartment presses 7404 so that they are pressed against the external surface of the fluid compartments. The latching arms 7400 not only secure the pump body to the terminal adapter, but also put the Resonator Probe Antennas 7000 in a position on the external surface so that the flow can be monitored. In an aspect, the pump body is a disposable element that can be discarded and replaced after each use or after a certain number of uses. Having the Resonator Probe Antennas 7000 integrated into the latching arms 7400 saves them from being a part of the disposable pump body, meaning that only the pump body need be replaced, while the Resonator Probe Antennas 7000 remain an integrated part of the terminal adapter and can be reused. In an aspect, closing the Resonator Probe Antennas 7000 against the fluid compartments puts the Resonator Probe Antennas 7000 in a position to monitor flow. In an aspect, the Resonator Probe Antennas 7000 monitor flow by sending out a signal from the transmit antenna through the dielectric fluid in the fluid compartment. Then, the signal passes back through the dielectric fluid and it is received by the receive antenna. The information from this process is received by a phase/gain measurement module and a controller. The changing volume in the fluid compartment causes a phase shift in the signals being transmitted by the receive antenna. This phase shift is analyzed by the controller and is used to determine the amount of fluid in the fluid compartment. The larger the phase shift, the larger the volume of fluid in the fluid compartment, and if there is no phase shift then there is no fluid in the fluid compartment. This information can be used to determine what stage of flow the system is in, indicating to a user if a stroke has ended or is continuing.

Still referring to FIG. 7, in another embodiment the integrated flow monitor is a low pass filter similar to the one described in FIGS. 3A-3B.

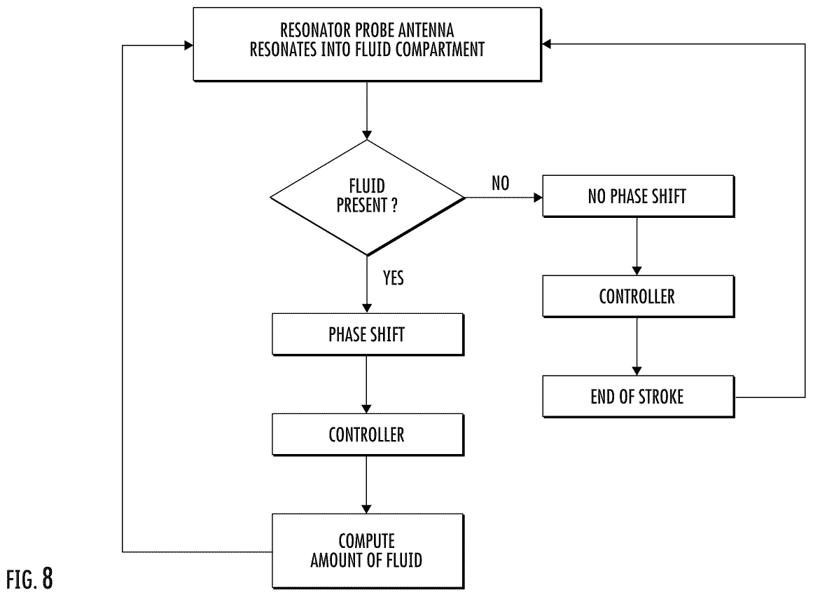

Referring now to FIG. 8, which illustrates a method of monitoring the flow of fluid. In an aspect, the method includes a Resonator Probe Antenna transmitting a signal into a fluid compartment. This for example, but limited by the example, may include having a Resonator Probe Antenna against the external surface of a fluid compartment and the transmit antenna of the Resonator Probe Antenna sends signals into the fluid compartment. The next order of events is determined by the presence or absence of fluid in the fluid compartment.

Still referring to FIG. 8. When fluid is present in the fluid compartment, phase shift occurs. This is because the dielectric fluid alters the phase of the signal, resulting in a phase shift when the receive antenna receives the antenna. In an aspect, this information may then be passed onto a controller, where the extent of the phase shift is used to compute the amount of fluid present in the fluid compartment. The greater the phase shift, the greater the amount of fluid present in the fluid compartment. This method will continue to occur starting again with the Resonator Probe Antenna transmitting a signal into the fluid compartment.

Still referring to FIG. 8. When no fluid is present in the fluid compartment, no phase shift occurs. This is because there is no dielectric fluid to alter the phase of the signal that would result in a phase shift when the receive antenna receives the antenna. In an aspect, this information may then be passed onto a controller, where the absence of phase shift is used to determine that there is no fluid present and an end of stroke has occurred. This method will continue to occur, starting again with the Resonator Probe Antenna transmitting signal into the fluid compartment.

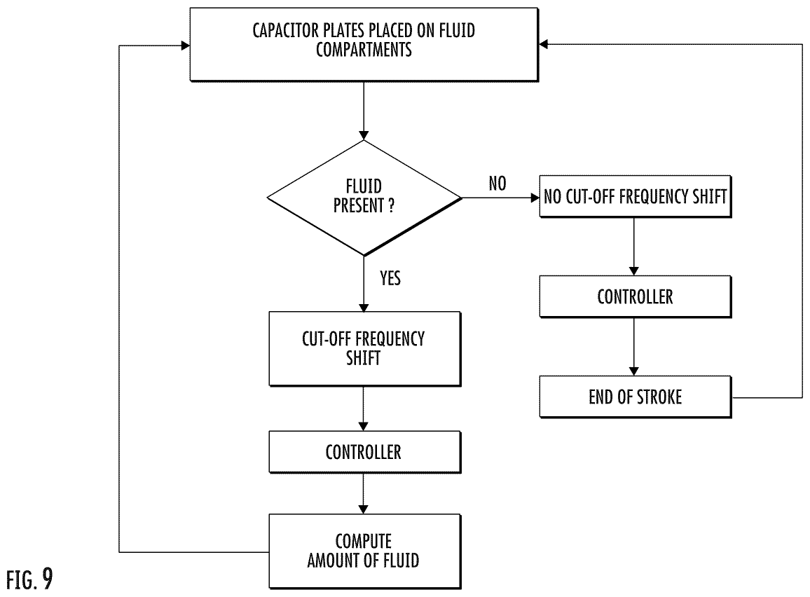

Referring now to FIG. 9, which illustrates a method of monitoring the flow of fluid. In an aspect, the method includes forming a capacitor in conjunction with a fluid compartment. This for example, but not limited by the example, may include placing capacitor plates on the external surface of the fluid compartments and connecting said capacitor plates with an inductor. This may also include connecting said capacitor plates to a capacitor ground plate that is also on the external surface of the fluid compartments. A low-pass filter capacitor is then formed between the capacitor plates, inductor, capacitor ground plate and the contents of the fluid compartment (a dielectric fluid) serve as the dielectric element of the capacitor. The next order of events is determined by the presence or absence of fluid in the fluid compartment.

Still referring to FIG. 9. When fluid is present in the fluid compartment, phase shift occurs. This is because the volume of dielectric fluid is changing, and the dielectric fluid serves as the dielectric element of the capacitor. As the dielectric element to the capacitor, when the volume of dielectric fluid in the fluid compartment changes, the capacitance of the capacitor changes. The change in capacitance results in the low-pass filter cut-off frequency changing. The change in fluid volume and subsequent change in low-pass filter cut-off frequency change results in a phase shift. In an aspect, this information may then be passed onto a controller, where the extent of the phase shift is used to compute the amount of fluid present in the fluid compartment. The greater the phase shift, the greater the amount of fluid present in the fluid compartment. This method will continue to occur starting again with the low-pass filter being grounded and connected by an inductor and having a certain capacitance based on the amount of liquid.

Still referring to FIG. 9. When no fluid is present in the fluid compartment, no cut-off frequency shift occurs. When no cut-off frequency shift occurs, the phase shift is much less than when cut-off frequency shift does occur. In an aspect, this information may then be passed onto a controller, where the absence of band width cut-off shift is used to determine that there is no fluid present and an end of stroke has occurred. This method will continue to occur starting again with the low-pass filter being grounded and connected by an inductor and having a certain capacitance based on the amount of liquid.

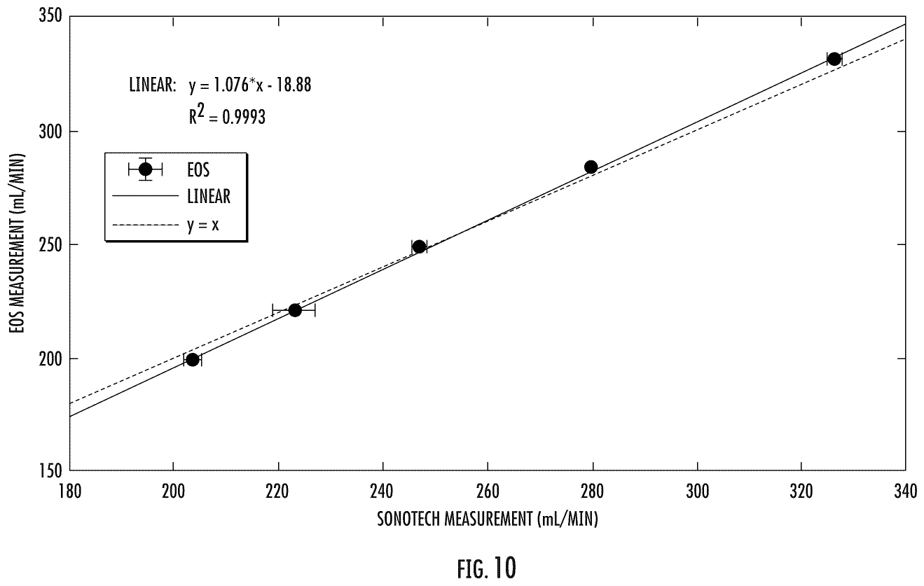

Referring now to FIG. 10, which is a graph depicting the accuracy of a flow monitor, wherein the flow monitor is a resonator probe antenna as described in FIG. 1, or a low-pass filter as described in FIG. 3A-3B, against a commercial in-line flow sensor. From the graph it can be seen that in an aspect, using a flow monitor to measure fluid is highly accurate, similar to that of a much more expensive commercial in-line flow sensor.

While the principles of the invention have been described herein, it is to be understood by those skilled in the art that this description is made only by way of example and not as a limitation as to the scope of the invention. Other embodiments are contemplated within the scope of the present invention in addition to the exemplary embodiments shown and described herein. Modifications and substitutions by one of ordinary skill in the art are considered to be within the scope of the present invention.

What is claimed is:

1. A system for monitoring fluid flow, the system comprising:
a Resonator Probe Antenna arranged on an external surface of a fluid compartment,
the Resonator Probe Antenna having:
a transmit antenna;
a receive antenna;
at least one electrical connector;
a ground plate; and
a controller communicatively coupled to said Resonator Probe Antenna;
wherein said controller transmits a signal having a first phase via said transmit antenna,
wherein said controller receives said signal at said receive antenna,
wherein said controller detects a phase shift in said signal as between said transmitting and receiving.

2. The system of claim 1, wherein a volume of fluid flows through said fluid compartment.

3. The system of claim 2, wherein said fluid is a dielectric fluid.

4. The system of claim 2, wherein said transmit antenna transmits a signal into said fluid compartment.

5. The system of claim 4, wherein said fluid volume has an effect on said signal causing said phase shift.

6. The system of claim 5, wherein said receive antenna receives said effected signal.

7. The system of claim 6, wherein said controller analyzes said phase shift and determines a stage of flow.

8. The system of claim 7, wherein the stage of flow is an end of stroke.

9. The system of claim 7, wherein the stage of flow is a current stroke.

10. The system of claim 9, wherein the current stroke has a varying fluid volume.

11. The system of claim 10, wherein said controller determines a quantity of said varying fluid volume.

12. The system of claim 2, wherein said fluid compartment is a part of a pump body.

13. The system of claim 12, wherein said pump body is fluidly connected to a fluid source, said fluid source pumps said fluid volume into said fluid compartment.

14. The system of claim 1, wherein a cable provides said communicative coupling between the at least one electrical connector and the controller.

15. The system of claim 1, wherein the fluid compartment is disposable.

16. The system of claim 1, wherein the Resonator Probe Antenna is disposable.

17. The system of claim 12, wherein the pump body is disposable.

18. A system for monitoring fluid flow, the system comprising:
a low-pass filter arranged on an external surface of a fluid compartment, the low-pass filter having:
at least one capacitor plate;
at least one electrical connector;
a capacitor ground plate
an inductor; and
a controller communicatively coupled to said low-pass filter;
wherein said controller detects a first capacitance based on a first state of fluid flow,
wherein said controller detects at least one second capacitance based on at least a second state of fluid flow,
wherein a difference between said first capacitance and said at least second capacitance indicate a phase shift.

19. The system of claim 18, wherein a fluid volume of a fluid flows through said fluid compartment.

20. The system of claim 19, wherein said fluid is a dielectric fluid.

21. The system of claim 19, wherein said low-pass filter forms a capacitor including a volume of fluid within said fluid compartment and has said capacitance.

22. The system of claim 21, wherein said fluid volume has an effect on said capacitance causing said phase shift.

23. The system of claim 18, wherein said controller analyzes said phase shift and determines a stage of flow.

24. The system of claim 23, wherein said stage of flow is an end of stroke.

25. The system of claim 23, wherein said stage of flow is a current stroke.

26. The system of claim 25, wherein said current stroke has a varying fluid volume.

27. The system of claim 26, wherein said controller can determine a value of said varying fluid volume.

28. The system of claim 19, wherein said fluid compartment is a part of a pump body.

29. The system of claim 28, wherein said pump body is fluidly connected to a fluid source, said fluid source pumps said fluid volume into said fluid compartment.

30. The system of claim 18, wherein a cable provides said communicative coupling between the at least one electrical connector and the controller.

31. The system of claim 18, wherein the fluid compartment is disposable.

32. The system of claim 18, wherein the low-pass filter is disposable.

33. The system of claim 28, wherein said pump body is disposable.
